(19) United States
(12) Patent Application Publication
(10) Pub. No.: US 2009/0325175 A1
(43) Pub. Date: Dec. 31, 2009

(54) METHODs OF DETECTION AND QUANTIFICATION OF HOST CELL DNA CONTAMINATION OF PURIFIED PROTEINS

(75) Inventors: Karin Anderson, Georgetown, MA (US); Mark William Leonard, Manchester, NH (US); Denise Alice Fahey, Deerfield, NH (US)

Correspondence Address: WYETH;PATENT LAW GROUP, 5 GIRALDA FARMS, MADISON, NJ, 07940, US

(73) Assignee: WYETH, MADISON, NJ (US)

(21) Appl. No.: 12/476,101

(22) Filed: Jun. 1, 2009

Related U.S. Application Data
(60) Provisional application No. 61/057,723, filed on May 30, 2008.

(51) Int. Cl. C12Q 1/68

(52) U.S. Cl. 435/6

(57) ABSTRACT

The present invention provides a novel robust, sensitive, reproducible, and accurate method of detecting and quantifying host cell genomic DNA contamination utilizing quantitative real time Polymerase Chain Reaction (qPCR), wherein the qPCR primers are complementary to the highly repetitive host cell genomic DNA sequences, e.g., Alu-equivalent sequences. The present invention is particularly useful for determining the levels of residual genomic DNA in biological products to be administered as therapeutics, e.g., therapeutic proteins.

### RELATED APPLICATIONS

This application claims the benefit of priority from U.S. Provisional Application No. 61/057,723, filed May 30, 2008, the content of which is hereby incorporated by reference herein in its entirety.

### BACKGROUND OF THE INVENTION

1. Field of the Invention

The present invention relates to methods of detection and quantification of nucleic acid contamination in various stages of protein purification. More specifically, the present invention relates to the method of detecting and quantifying host cell genomic DNA contamination utilizing quantitative real time Polymerase Chain Reaction (qPCR), wherein the qPCR primers are complementary to the highly repetitive host cell genomic DNA sequences. Thus, the present invention allows robust, sensitive, reproducible and accurate detection and quantification of DNA in a purified biological product to be administered as a therapeutic, e.g., a purified therapeutic protein, e.g., a purified therapeutic antibody, etc.

2. Related Background Art

The World Health Organization (WHO) and the Food and Drug Administration (FDA) recommend that pharmaceutical products, e.g., pharmaceutical products comprising proteins, contain no more than 10 ng of residual host DNA per dose of protein. The FDA further recommends using detection methods having sensitivity of at least 10 picograms per dose of protein (see, Points to Consider in the Characterization of Cell Lines Used to Produce Biologicals, Office of Biologics Research and Review, FDA (revised May 1993)).

Several methods are currently used for determining the presence and levels of residual DNA in pharmaceutical products. For instance, THRESHOLD™ Total DNA Assay System (Molecular Devices Corporation, Menlo Park, Calif.) uses an automated reader to quantify DNA by detecting the rate of pH change in enzyme-bound DNA samples. This method is disadvantageous because it is costly, labor intensive and restrictive (in that specific compatible buffers must be chosen).

Other methods of determining the levels of DNA contamination utilize Polymerase Chain Reaction (PCR). Goldman et al. (Clinical Chemistry, 37:1523 (1991)) describe a method by which primers to E. coli 16S ribosomal RNA are used to detect E. coli DNA contamination. However, 16S ribosomal RNA and other genes used for genomic-contamination detection are present in the host cell in relatively low copy, making detection of DNA on the order of picograms difficult.

Letwin and Jezuit (U.S. Pat. No. 5,393,657) describe the use of primers to repetitive DNA sequences scattered throughout the genome for residual DNA detection. Primers to repetitive DNA sequences, e.g., Alu-equivalent consensus sequences, were used to amplify residual CHO cell genomic DNA in a sample. However, detection and quantification of contaminants relied on conventional molecular biology techniques, such as gel electrophoresis, Southern Blot, DNA monoclonal antibodies, cloning, sequencing, etc. Although U.S. Pat. No. 5,393,657 provides useful techniques for detecting contaminant genomic DNA, there exists a need for an alternative high-throughput method of detection and quantification of residual genomic DNA in pharmaceutical products.

### SUMMARY OF THE INVENTION

One embodiment of the invention provides a method of detecting contaminant genomic DNA in a sample comprising: purifying genomic DNA from the sample; adding a pair of oligonucleotide primers that are complementary to repetitive sequences of genomic DNA; amplifying the repetitive sequences with the pair of oligonucleotide primers using a real time PCR amplification method; and detecting the presence of amplified repetitive sequences, wherein the detection of the amplified repetitive sequences indicates the presence of the contaminant genomic DNA in the sample. In at least one embodiment, the invention provides a method wherein the real time PCR amplification method is a quantitative real time PCR amplification method. Preferably, the quantitative real time PCR amplification method utilizes TAQMAN® Probe Technology.

Another embodiment of the invention provides a method of detecting contaminant genomic DNA in a sample comprising: purifying genomic DNA from the sample; adding both a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on the opposite end; amplifying the repetitive sequences using a nucleic acid polymerase having 5′ to 3′ exonuclease activity; and measuring the change in fluorescence of the sample during amplification, wherein the change in fluorescence indicates detection of amplified repetitive sequences and correlates with the presence of the contaminant genomic DNA in the sample. In some embodiments of the invention, the step of purifying genomic DNA from the sample comprises: digesting protein and RNA in the sample, and extracting total DNA from the sample by precipitation. In a preferred embodiment, the step of purifying genomic DNA from the sample comprises using MASTERPURE™ DNA Purification Kit.

In one embodiment of the invention, the pair of oligonucleotide primers used in the method of the invention comprises the nucleic acid sequence of SEQ ID NO:2 and SEQ ID NO:3. In another embodiment, the oligonucleotide probe comprises nucleic acid sequences selected from the group consisting of SEQ ID NO:4, SEQ ID NO:10, SEQ ID NO:11, SEQ ID NO:12, and SEQ ID NO:13. In a preferred embodiment, the oligonucleotide probe comprises nucleic acid sequence of SEQ ID NO:4.

In some embodiments, the invention utilizes repetitive sequences that are either Alu sequences or Alu-equivalent sequences. In another embodiment, the invention utilizes the pair of oligonucleotide primers and the oligonucleotide probe that are designed based on a consensus of several Alu sequences or Alu-equivalent sequences of an organism.

Moreover, in some embodiments of the invention, the fluorescent reporter utilized is FAM and the quencher dye is TAMRA. In further embodiments, the nucleic acid polymerase utilized is Taq polymerase. In some embodiments of the invention, the genomic DNA is a CHO cell genomic DNA.

In some embodiments, the step of amplifying the repetitive sequences further comprises a step of monitoring for sample recovery and assay performance, wherein the step of monitoring comprises adding a known genomic DNA spike to the sample. In one embodiment, the known genomic DNA spike has a genomic DNA concentration of 10 ng/mL.

In one embodiment of the invention, the invention provides a method wherein the sample comprises a purified protein. In a further embodiment, the sample is a pharmaceutical composition, and the purified protein is a therapeutic protein.

In yet another embodiment of the invention, a method of quantifying contaminant genomic DNA in a first sample comprises: purifying genomic DNA from the first sample; adding to the first sample a pair of oligonucleotide primers that are complementary to repetitive sequences of the genomic DNA; adding to a second sample, comprising a known amount of genomic DNA, a pair of oligonucleotide primers that are complementary to repetitive sequences of the genomic DNA; amplifying repetitive DNA sequences in the first and second samples using a real time PCR amplification method; and determining from the amplified repetitive DNA sequences of the second sample the amount of the contaminant genomic DNA in the first sample. In one embodiment, the invention provides a method wherein the real time PCR amplification method is a quantitative real time PCR amplification method. Preferably, the quantitative real time PCR amplification method is TAQMAN® Probe Technology.

An additional embodiment of the invention provides a method of quantifying contaminant genomic DNA in a first sample comprising: purifying genomic DNA from the first sample; adding to the first sample a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on an opposite end; adding to a second sample, comprising a known amount of genomic DNA, a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on an opposite end; amplifying repetitive DNA sequences in the first and second samples using a nucleic acid polymerase having 5′ to 3′ exonuclease activity; measuring the change in fluorescence of the first and second samples during amplification; comparing the change in fluorescence of the first and second samples; and determining from the comparison of fluorescence of the first and second samples the amount of contaminant genomic DNA in the first sample. In some embodiments of the invention, the step of purifying genomic DNA from the first sample comprises: digesting protein and RNA in the sample, and extracting total DNA from the sample by precipitation. In a preferred embodiment, the step of purifying genomic DNA from the sample comprises using MASTERPURE™ DNA Purification Kit.

In one embodiment of the invention, the pair of oligonucleotide primers used in the method of the invention comprises the nucleic acid sequence of SEQ ID NO:2 and SEQ ID NO:3. In another embodiment, the oligonucleotide probe comprises nucleic acid sequences selected from the group consisting of SEQ ID NO:4, SEQ ID NO:10, SEQ ID NO:11, SEQ ID NO:12, and SEQ ID NO:13. In a preferred embodiment, the oligonucleotide probe comprises nucleic acid sequence of SEQ ID NO:4.

In some embodiments, the invention utilizes repetitive sequences that are either Alu sequences or Alu-equivalent sequences. In another embodiment, the invention utilizes the pair of oligonucleotide primers and the oligonucleotide probe that are designed based on a consensus of several Alu sequences or Alu-equivalent sequences of an organism.

Moreover, in some embodiments of the invention, the fluorescent reporter utilized is FAM and the quencher dye is TAMRA. In further embodiments, the nucleic acid polymerase utilized is Taq polymerase. In some embodiments of the invention, the genomic DNA is a CHO cell genomic DNA. In some embodiments, the known amount of genomic DNA in the second sample is predigested with Msp I and Kpn I restriction enzymes.

In some embodiments of the invention, the first sample comprises a purified protein. In a further embodiment, the sample is a pharmaceutical composition, and the purified protein is a therapeutic protein. In another further embodiment, the amount of contaminant genomic DNA in the pharmaceutical composition is less than 10 nanograms per dose.

### BRIEF DESCRIPTION OF THE DRAWINGS

FIG. 1 represents the alignment of the four cloned Alu-equivalent sequences (designated as “100_{—}10.ed,” “50_{—}10RS.ed,” “500.ed,” and “500pRS10_{—}1.ed,” which are represented in SEQ ID NOs:5, 6, 7, and 8, respectively) and the nucleotides 6-84 (designated as “Haynes consensus”; SEQ ID NO:1) of the Alu-equivalent consensus sequence from Haynes et al. ((1981) Mol. Cell. Biology 1:573-83). “Majority” (SEQ ID NO:9) represents a new Alu-equivalent consensus sequence derived from the alignment.

FIG. 2 represents optimization of performance (FIG. 2B) and sensitivity (FIG. 2C) of degenerate and nondegenerate probes and indicated combinations of nondegenerate probes at 200 nM concentration in each reaction. The probes were designed based on Alu sequence analysis (FIG. 2A). FIG. 2D represents assessment of performance of probes C and D (SEQ ID NO:4 and SEQ ID NO:12, respectively), or a combination thereof, at different probe concentrations.

FIG. 3 represents the comparison between Qiagen and Gentra DNA extraction methods: standard curves for both methods shown in FIG. 3A; average of three threshold cycle (C_{T}) replicates of extracted buffer (“buffer”), no-template control (“NTC”), and the last point on the curve (“0.01 pg DNA/PCR well”) compared in FIG. 3B; and spike recovery results of samples spiked with either 50 pg or 50 ng of CHO genomic DNA, expressed as a log difference between the expected and observed values, shown in FIG. 3C. “UD” indicates values below the level of detection (undetectable).

FIG. 4 represents the comparison between Gentra and EpiCentre DNA extraction methods, with standard curves for both methods shown in FIG. 4A; average of three threshold cycle (C_{T}) replicates of extracted buffer (“buffer”), no-template control (“NTC”), and the last point on the curve (“0.1 pg DNA/PCR well”) compared in FIG. 4B; and spike recovery results of two samples spiked with 50 pg CHO genomic DNA, expressed as a log difference between the expected and observed values, shown in FIG. 4C. “UD” indicates values below the level of detection (undetectable).

FIG. 5 is a flow chart representing alterations of the EpiCentre DNA extraction method that improves DNA recovery; main alterations are indicated in bold.

### DETAILED DESCRIPTION OF THE INVENTION

The present invention provides a robust, sensitive, reproducible and accurate method of detection and quantification of contaminant genomic DNA. The method comprises (1) preparation of residual genomic DNA, i.e., DNA separated from other sample components (protein, buffer components that are inhibitory to the assay, etc.) and (2) quantitative real time PCR (qPCR) assay utilizing primers to Alu consensus sequences or Alu-equivalent consensus sequences.

The phrases “contaminant genomic DNA,” “residual genomic DNA,” and the like refer to any nucleic acid molecules remaining in the sample, e.g., purified protein sample, pharmaceutical formulation, etc. Nucleic acid molecules remaining in the sample can comprise apoptotic DNA fragments, i.e., fragments of DNA resulting from programmed cell death.

The sample tested for the presence of residual genomic DNA by the method of the present invention can be any sample for which detection and quantification of DNA contamination is required, e.g., a polypeptide purification fraction at any stage of protein purification, a final drug formulation, a pharmaceutical composition containing the polypeptide to be administered as a pharmaceutical agent, etc.

### Preparation of Residual Genomic DNA

Various methods of preparation of residual genomic DNA can be used in the methods of the present invention. In the present invention, preparation of residual genomic DNA (DNA sample preparation) comprises digesting the protein and RNA in the sample, extracting and precipitating DNA, and resuspending the DNA pellet in solution, e.g., water. Typically, a proteinase, e.g., Proteinase K, is used to digest the proteins contained in a sample, while an RNase, e.g., RNase A, is used to digest the RNA. Remaining DNA is subsequently purified, e.g., by precipitation. For instance, MASTERPURE™ DNA Purification Kit (EPICENTRE®, Madison, Wis.) uses a nontoxic desalting method (U.S. Pat. No. 6,270,962) to purify DNA from any contaminants. One skilled in the art will know that including, e.g., yeast transfer RNA (tRNA), in the purification reaction may prevent nonspecific adsorption or loss of DNA.

Various methods of DNA purification are known in the art and may be used in the methods of the invention. However, some of the known methods of DNA purification are not efficient in recovering DNA fragments smaller than 180 base pairs, e.g., apoptotic DNA fragments. Moreover, some methods, e.g., QIAamp DNA Kit (Qiagen, CA), may involve tube handling, e.g., transferring material between two or more tubes, which pose a risk of sample loss and contamination; thus providing inaccurate amounts of contaminant genomic DNA in a sample, e.g., in a pharmaceutical composition. Less efficient methods of DNA purification involve, for example, DNA purification in an ion-exchange column; whereas more efficient methods of DNA purification involve, for example, DNA precipitation, e.g., MASTERPURE™ DNA Purification Kit. One skilled in the art will know which methods of DNA purification are equally efficient to MASTERPURE™ DNA Purification Kit in DNA recovery and do not pose a risk of sample loss and contamination.

### Detection and Quantification of Residual Genomic DNA

The methods of the present invention use qPCR to detect and quantify residual genomic DNA in a sample.

PCR is a method for rapid nucleic acid amplification that is well known in the art (see, e.g., U.S. Pat. Nos. 4,683,195; 4,683,202; and 4,965,188). PCR generally comprises adding DNA polymerase, dNTPs, buffer, oligonucleotides (primers, e.g., a pair of oligonucleotide primers), to the sample (template), and subjecting this PCR master mix to at least one cycle comprising the steps of denaturing (melting), annealing (or hybridizing), and elongating (or extending). One skilled in the art will recognize that the denaturing, annealing, and elongating steps of PCR may be effectuated by altering the temperature of the PCR mixture. One of skill in the art will also recognize that the temperatures, the length of time at such temperatures, and the number of PCR cycles that the sample must be subjected to will differ for different primers.

Quantitative real time PCR (qPCR) is a quantitative method of measuring the nucleic acid products generated during each PCR cycle, wherein the amount of nucleic acid products generated is a measure of the amount of nucleic acid present in the sample at the start of a PCR reaction. In the methods of the present invention, qPCR can be used to detect and quantify small amounts of purified residual genomic DNA in a sample. There are many benefits of using the qPCR assay in the methods of the present invention, e.g., broad dynamic range, relatively low inter- and intra-assay variability, high data reproducibility, and robustness.

In the present invention, detection and quantification of residual genomic DNA comprises using primers, e.g., a pair of oligonucleotide primers, to repetitive DNA sequences. For instance, Alu-repetitive DNA sequences comprise about 6-13% of human genomic DNA, about 100,000 to 1 million copies in the human genome (see, e.g., Rowald and Herrera (2000) Genetics 108:57-72). The Alu family of repetitive DNA sequences is present in most or all mammalian genomes, including human, mouse, hamster (e.g., CHO cells), etc. Alu sequences are named after the AluI restriction enzyme site within the consensus Alu sequence. Properties and the molecular origin of Alu-repetitive sequences are described in Mighell et al. (199.7) FEBS Lett. 417:1-5. Other repetitive DNA sequences are described in the literature and can be used to design primers for the methods of the present invention. For example, primers directed to minisatellite DNA sequences may be used in the present invention. Minisatellite regions of human DNA are described in Jeffreys et al. (1985) Nature 313:67-72 and Wong et al. (1986) Nucleic Acids Res. 14:4605-15. Other repetitive DNA sequences are described in, e.g., Jelinek and Schmid (1982) Ann. Rev. Biochem. 51:813-44; Moyziz et al. (1989) Genomics 4:273-89; Lupski and Weinstock (1992) J. Bacteriol. 174:4525-29; Sharples (1990) Nucleic Acids Res. 18:6503-08; Eisenach et al. (1990) J. Infect Dis. 161:977-81. Therefore, primers directed to repetitive DNA sequences, e.g., Alu, IRU (intergenic repeat unit), REP (repetitive extragenic palindrome), L1, etc., may be used to amplify contaminant genomic DNA of the present invention.

Depending on the cell type used for expression of the polypeptide and/or preparation of the pharmaceutical composition, the repetitive sequences may vary. Thus, as used herein, “Alu sequences” refers to Alu repetitive genomic DNA sequences in any primate mammalian organism or cell type. “Alu-equivalent sequences,” as used herein, refers to repetitive ALE sequences found in mammals other than primates, wherein the Alu-equivalent sequences exhibit the same properties as the primate Alu sequences. However, there may be slight variations in Alu sequences or Alu-equivalent sequences between different representative cell clones of a particular cell type, or between different representative organisms of the same species. Thus, as used herein, “Alu consensus sequences” or “Alu-equivalent consensus sequences” refers to sequences derived from multiple sequence alignment of different Alu sequences or Alu-equivalent sequences obtained from the same species or the same cell type (either primate mammals or non-primate mammals, respectively), wherein the Alu consensus sequences or the Alu-equivalent consensus sequences comprises the sequence of nucleotides in common or most common between the represented clones. For example, SEQ ID NO:1 is the CHO cell Alu-equivalent consensus sequence based on Haynes et al. ((1981) Mol. Cell. Biology 1:573-83).

In the methods of the present invention, a primer refers to an oligonucleotide, e.g., a nucleic acid polymer of at least two nucleic acid residues, preferably more than 20 nucleic acid residues, which is complementary to the nucleotide sequence representing an Alu sequence or an Alu consensus sequence (or alternatively, an Alu-equivalent sequence or an Alu-equivalent consensus sequence). As used herein, “complementary” refers to an oligonucleotide derived from the sequence of, and/or substantially identical to, either the sense (+) or antisense (−) strand of residual genomic DNA, e.g., substantially identical to the sense or antisense strand of the nucleic acid sequence representing an Alu sequence or an Alu-equivalent sequence (or alternatively, an Alu consensus sequence or an Alu-equivalent consensus sequence). For example, a primer having a nucleotide sequence complementary to the Alu-equivalent consensus sequence is capable of annealing (or hybridizing) to either the sense or antisense strand on the residual genomic DNA under stringent conditions. SEQ ID NO:2 and SEQ ID NO:3 represent primers complementary to the sense and antisense strands, respectively, of the CHO cell Alu-equivalent consensus sequence. Such primers are used to amplify residual genomic DNA in a qPCR reaction.

Various methods of qPCR can be used with the methods of detecting and quantifying the residual genomic DNA of the present invention. In one instance, the qPCR method used is a hydrolysis probe method, such as the TAQMAN® probe technology. This method utilizes Fluorescence Resonance Energy Transfer (FRET) principles to detect and quantify residual genomic DNA.

In addition to the two primers, e.g., the sense and the antisense primers, the TAQMAN® probe technology utilizes a fluorogenic nonextendable probe (TAQMAN® probe) that is complementary to the sequence 3′ of one of the primers. The probe is capable of hybridizing to the nucleic acid sequence (the template sequence, e.g., repetitive sequence of genomic DNA) 3′ relative to one of the two primers. The probe contains a fluorescent reporter dye attached to its 5′ end, e.g., 6-carboxyfluorescein (FAM), tetrachloro-6-carboxy-fluorescein (TET), hexachloro-6-carboxyfluorescein (HEX), etc., and a quencher dye at its 3′ end, e.g., 6-caboxytetramethylrhodamine (TAMRA), 4-(dimethylaminoazo)benzene-4-carboxylic acid (DABCYL), etc. In a preferred embodiment of the invention, the fluorescent reporter dye is FAM and the quencher dye is TAMRA. During the annealing step of the PCR, the probe anneals to the target nucleotide sequence, e.g., the Alu-equivalent consensus sequence. In such configuration, the fluorescence of the reporter dye at the 5′ end of the TAQMAN® probe is quenched by the quencher at the 3′ end of the probe. SEQ ID NO:4 represents a TAQMAN® probe complementary to the CHO cell Alu-equivalent consensus sequence of the invention.

The probe complementary to the CHO cell Alu-equivalent consensus sequence can be a degenerate probe or a nondegenerate probe. A nondegenerate probe is a probe in which nucleic acid residues at all positions are selected from A, T, G, or C. A degenerate probe is a probe in which nucleic acids are not defined at all positions, rather these probes allow more than one nucleic acid to be incorporated into the elongating nucleic acid at one or more position(s). For instance, Y indicates that any pyrimidine, i.e., T and C, is allowed at a particular position; R indicates that any purine, i.e., A and G, is allowed at a particular position; H indicates that any one of C, T, and A is allowed at a particular position; and N indicates that any one of A, T, C, and G is allowed at a particular position. Because different cell clones may possess slightly different Alu-equivalent sequences, degenerate probes may allow detection of Alu-equivalent sequences from different CHO cell clones. In one embodiment of the invention, a degenerate probe for an Alu-equivalent consensus sequence is represented by SEQ ID NO:13, and nondegenerate probes for an Alu-equivalent consensus sequence are represented by SEQ ID NOs:4, 10, 11, and 12.

A preferred probe of the invention is highly sensitive, has a broad dynamic range, and has a high signal-to-background ratio. A skilled artisan will recognize that sensitivity refers to the ability of the probe to detect residual host DNA, preferably less than 10 pg residual host DNA, more preferably less than 0.1 pg residual host DNA, most preferably 0.01 pg residual host DNA, in the least number of PCR cycles, e.g., less than 30 cycles of PCR. A skilled artisan will also recognize that broad dynamic range refers to the ability of the probe to detect variable amounts of residual genomic DNA, e.g., amounts of residual genomic DNA that vary by several logarithmic units, e.g., 1000 to 0.01 pg. The high signal-to-background ratio refers to the specificity of the probe; preferred probes of the invention should be specific for the Alu sequence or the Alu-equivalent sequence and not recognize any other sequence in the genomic DNA.

TAQMAN® probe technology uses Taq DNA polymerase, e.g., AMPLITAQ® Gold DNA polymerase, which has the 5′ to 3′ exonuclease activity. During the elongation step of the PCR, the TAQMAN® probe is cleaved by the Taq DNA polymerase, separating the reporter and quencher dyes, such that the fluorescence of the reporter is no longer quenched, and the change in fluorescence can now be detected and monitored. In embodiments of the present invention, a change in fluorescence indicates detection of amplified repetitive sequences and correlates with the presence of contaminant genomic DNA. Fluorescence emission is detected in real time by the thermocycler. For example, if the TAQMAN® ABI Prism 7000 (BioReliance, MD) instrument is used, the ABI Prism 7000 software also is used to measure fluorescent signal intensity of each sample. Inclusion of the known amount of control DNA, e.g., a 10 ng/mL CHO genomic DNA, provides a control for sample recovery and assay performance. Standards of known amounts of, e.g., DNA representing Alu consensus sequence or Alu-equivalent consensus sequence, are assayed in parallel to quantify the residual genomic DNA based on the standard curve.

One skilled in the art will know how to conduct a qPCR experiment and analyze the data. A typical qPCR reaction consists of at least one cycle, preferably more than about 25 cycles, and more preferably about 45 cycles of PCR. In one embodiment of the invention, qPCR reaction is a two step PCR reaction consisting of a melting step (denaturing step) at 95° C. for 15 seconds followed by the annealing/extending step at 60° C. for 1 minute. A skilled artisan would understand that various parameters of the qPCR reaction, e.g., the temperature and the length of each qPCR cycle, depend on various factors, including the length of the fragment, the composition of the primers, the length of the primers, etc.; thus, a skilled artisan will know how to adjust the parameters of the qPCR reaction.

In a qPCR reaction, computer software, e.g., ABI Prism 7000 software, constructs amplification plots using the fluorescence data collected during each cycle of PCR. On the amplification plot, baseline refers to the initial PCR cycles, wherein the fluorescent signal is detected but is below the limit of detection of the instrument, e.g., the TAQMAN® ABI Prism 7000 or the TAQMAN® ABI Prism 7500 instrument. The default baseline is set, e.g., between about cycles 4 and 16 of the PCR. The threshold is an arbitrary value that is usually calculated as ten times the standard deviation of the average signal of fluorescence during initial PCR cycles, and is usually set in the exponential amplification phase of the PCR signal. The exponential (geometric) amplification phase is the phase during which the amount of nucleic acid in the sample takes one PCR cycle to double. A fluorescent signal above threshold is considered a real signal, and the cycle at which fluorescence passes the threshold is the threshold cycle (C_{T}). One skilled in the art will know that a lower C_{T} for the reaction suggests that more nucleic acid was present in the sample at the start of the qPCR.

The “delta Rn” value is the difference between the fluorescence emission of the sample in the reaction and the fluorescence emission of the baseline. The delta Rn value is commonly plotted against cycle number of the reaction. One skilled the art will recognize that the delta Rn value will not exceed the baseline in the early cycles of qPCR. One skilled in the art will also recognize that C_{T} may be defined as the cycle at which the delta Rn crosses the threshold. Moreover, a skilled artisan will know that in the exponential amplification phase the delta Rn will increase; but as the sample components, e.g., dNTP, oligonucleotides, etc., become limited, the delta Rn will become constant, and the amplification curve for qPCR will plateau.

In the instant invention, the absolute amount of residual genomic DNA in the sample may be calculated based on the C_{T} values of the known standard sample. A known concentration of the standard is run in a qPCR reaction parallel to the unknown sample. Thus, the C_{T} values of the standard (Y-axis) can be graphed in a linear regression curve against the known initial DNA concentration of the standard (X-axis), and the amount of residual genomic DNA in the unknown sample can be extrapolated from the standard linear regression curve. In a preferred embodiment of the invention, the standard is a known concentration of CHO cell genomic DNA predigested with Msp I and Kpn I restriction enzymes. A detailed description of the qPCR methodology can be found in the Applied Biosystems manual entitled “Guide to Performing Relative Quantitation of Gene Expression Using Real-Time Quantitative PCR,” Part Number 4371095 (hereinafter “AB Guide”), and Arya et al. (2005) Expert Rev. Mol. Diagn. 5:209-19, each of which is hereby incorporated by reference in its entirety.

Various parameters are used to evaluate assay efficiency, assay precision, etc., based on the linear regression curve of the standard, e.g., CHO cell genomic DNA predigested with Msp I and Kpn I. One skilled in the art will know that amplification assay efficiency refers to the rate at which the standard is amplified, and is commonly reflected in the slope of the linear regression curve of the standard. Thus, the slope of the linear regression curve of the standard is preferably between about −3 and −3.6, most preferably about −3.26. One skilled in the art will also know that the assay precision is related to the standard deviation of the C_{T} values between replicate samples of the standard, and is represented by the R² value. The R² value is preferably greater than about 0.98, more preferably about 1.00. The % CV value is a coefficient of variance, which is used to measure the level of variability of the assay. The % CV values are preferably below about 5%. Detailed descriptions of various parameters and methods of evaluating the qPCR results are described in the aforementioned manual (AB Guide).

In addition to TAQMAN® probe technology, other methodologies of qPCR can be used in the methods of the present invention. These methodologies include, but are not limited to, dual hybridization probes, molecular beacons, scorpion probes, etc. See, e.g., Emig et al. (1999) Leukemia 13:1825-32; Tyagi and Kramer (1996) Nat. Biotechnol. 14:303-08; Whitcombe et al. (1999) Nature 17:804-07, incorporated herein in their entirety by reference.

One skilled in the art will recognize that in the methods of the present invention there exists a need for preventing contamination of sample from foreign DNA. For example, both DNA extraction and PCR steps can be conducted in restricted hood areas, e.g., separate DNA extraction and qPCR hoods. Additionally, one skilled in the art will know to use, e.g., PCR-grade water (e.g., DNase and RNase free water).

### Proteins Tested for Residual Genomic DNA

Methods of testing for residual genomic DNA of the present invention may be used with any essentially pure protein, including, but not limited to, essentially pure proteins having pharmaceutical, diagnostic, agricultural, and/or any of a variety of other properties that are useful in commercial, experimental, and/or other applications. In addition, an essentially pure protein can be a protein therapeutic, e.g., an antibody therapeutic. Namely, a protein therapeutic is a protein that has a biological effect on a region of the body on which it acts directly, or on a region of the body on which it remotely acts via intermediates.

Methods of testing for residual genomic DNA may be used with any therapeutic protein, such as pharmaceutically or commercially relevant enzymes, receptors, receptor fusions, soluble receptors, soluble receptor fusions, antibodies (e.g., monoclonal and/or polyclonal antibodies), antigen-binding fragments of an antibody, Fc fusion proteins, SMIPs, cytokines, hormones, regulatory factors, growth factors, coagulation/clotting factors, or antigen-binding agents. The above list of proteins is merely exemplary in nature, and is not intended to be a limiting recitation. One of ordinary skill in the art will know of other proteins that can be tested for residual genomic DNA in accordance with the present invention, and will be able to use methods disclosed herein with such proteins or protein formulations.

One skilled in the art will know how to obtain a protein sample that may be tested for genomic DNA contamination using the methods of the present invention. For instance, a skilled artisan will know how to produce the protein therapeutic of interest in the cell, e.g., CHO cell, and how to purify the protein therapeutic from the cell. Subsequently, the protein therapeutic may be tested for genomic DNA contamination by the present invention and, e.g., incorporated into a pharmaceutical composition.

### Pharmaceutical Compositions Tested for Residual Genomic DNA

Methods of testing for residual genomic DNA may also be used with a pharmaceutical composition, e.g., a pharmaceutical composition comprising a therapeutic protein(s). Pharmaceutical compositions, e.g., a pharmaceutical composition comprising a therapeutic protein(s), used in methods of testing for residual genomic DNA may be administered to a subject or may first be formulated for delivery by any available route including, but not limited to, e.g., parenteral (e.g., intravenous), intradermal, subcutaneous, oral, nasal, bronchial, ophthalmic, transdermal (topical), transmucosal, intrathecal, intraventricular, epidural, rectal, and vaginal routes. Pharmaceutical compositions typically include a purified protein expressed from a mammalian cell line, a delivery agent (e.g., a cationic polymer, peptide molecular transporter, surfactant, etc., as described above), in combination with a pharmaceutically acceptable carrier.

As used herein the language “pharmaceutically acceptable carrier” includes nontoxic materials that do not interfere with the effectiveness of the biological activity of the active ingredient(s), e.g., solvents, dispersion media, coatings, antibacterial and antifungal agents, isotonic and absorption delaying agents, and the like, compatible with pharmaceutical administration. The characteristics of the carrier will depend on the route of administration. Supplementary active compounds can also be incorporated into the compositions.

A pharmaceutical composition is formulated to be compatible with its intended route of administration. When the therapeutic protein produced according to one or more methods of the present invention is administered in an oral form, the pharmaceutical composition usually will be in the form of a solution or an elixir. A liquid carrier such as water, petroleum, oils of animal or plant origin, such as sesame oil, peanut oil (taking into consideration the occurrence of allergic reactions in the population), mineral oil, or soybean oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition contains from about 0.5 to 90% by weight of the binding agent, and preferably from about 1 to 50% by weight of the binding agent.

When the pharmaceutical composition is administered by intravenous, cutaneous or subcutaneous injection, the pharmaceutical composition will be in the form of a pyrogen-free, parenterally acceptable solution. The preparation of such parenterally acceptable protein solutions, having due regard to pH, isotonicity, stability, and the like, is within the skill of those in the art. A preferred pharmaceutical composition for intravenous, cutaneous, or subcutaneous injection should contain, in addition to the therapeutic protein, an isotonic vehicle such as sodium chloride injection, Ringer's injection, dextrose injection, dextrose and sodium chloride injection, lactated Ringer's injection, or other vehicle as known in the art. The pharmaceutical composition may also contain stabilizers, preservatives, buffers, antioxidants, or other additive known to those of skill in the art.

All aforementioned formulations of the pharmaceutical compositions may be tested for residual genomic DNA using the methods of the present invention. Additional formulation of the pharmaceutical compositions comprising the therapeutic proteins that may be tested for residual genomic DNA using the methods of the present invention will be known to those skilled in the art. One of ordinary skill in the art will also be aware of unit dosage formulations appropriate for various pharmaceutical compositions.

Even though the invention has been described with a certain degree of particularity, it is evident that many alternatives, modifications, and variations will be apparent to those skilled in the art in light of the disclosure. Accordingly, it is intended that all such alternatives, modifications, and variations, which fall within the spirit and scope of the invention, be embraced by the defined claims.

The entire contents of all references, patents, and patent applications cited throughout this application are hereby incorporated by reference herein.

### EXAMPLES

The Examples which follow are set forth to aid in the understanding of the invention but are not intended to, and should not be construed to, limit its scope in any way. The Examples do not include detailed descriptions of conventional methods, such as PCR and gel electrophoresis, or those methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

Embodiments of the invention are discussed herein. The following Examples provide illustrative embodiments of the invention and do not in any way limit the invention. One of ordinary skill in the art will recognize that numerous other embodiments are encompassed within the scope of the invention.

### Example 1

### Method Development and Optimization

### Example 1.1

### Materials and Methods

The probe and primers were obtained from Integrated DNA Technologies (Coralville, Iowa). ABI Prism 7000 TAQMAN® Instrument and software were obtained from Applied Biosystems (San Diego, Calif.). PCR reagents for qPCR were obtained from TAQMAN® PCR Core Reagent Kit (Applied Biosystems). Quantitative real time PCR reactions were run for 45 cycles in a two-step PCR reaction consisting of a melting step (denaturing step) at 95° C. for 15 minutes, followed by the annealing/extension step at 60° C. for 1 minute.

### Example 1.2

### Determination of Alu-Equivalent Consensus Sequence

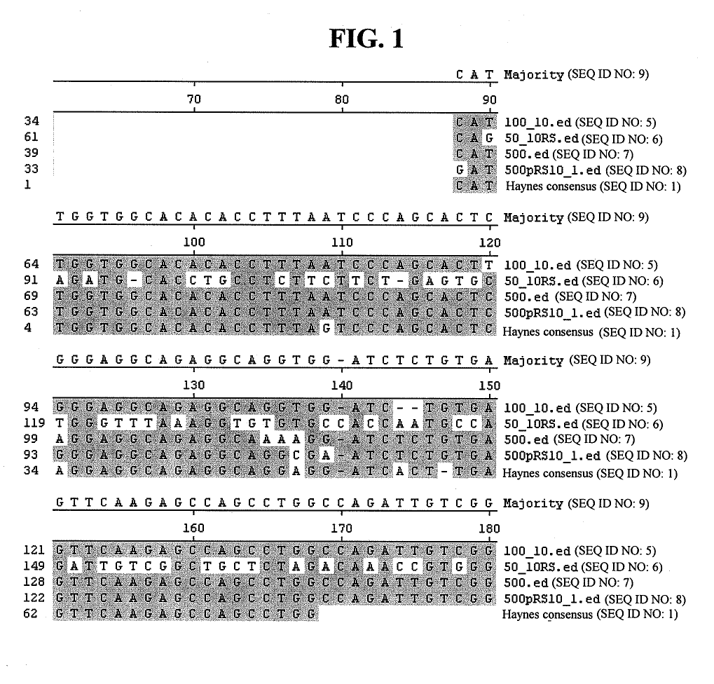

The target for the Chinese Hamster Ovary (CHO) cell DNA qPCR assay was the CHO Alu-equivalent consensus sequences described by Haynes et al., supra. Based on the 134 bp consensus sequence of six Alu-equivalent sequences cloned from CHO cells in Haynes et al., forward and reverse primers were designed. These were the same primers later utilized for the qPCR reaction (SEQ ID NO:2 and SEQ ID NO:3). PCR products with the expected size of 80 bp were generated using a high fidelity Taq Polymerase Pfu (Stratagene, La Jolla, Calif.). Ten PCR reactions were pooled after confirming the presence of a single band with the expected 80 bp size, and subcloned into a high copy E. coli vector using blunt end cloning kit (Invitrogen, Carlsbad, Calif.), transformed into ONE SHOT® chemically competent E. coli cells (Invitrogen) and plated on selective plates. Ten individual colonies were picked and the plasmids purified. Of these ten, five were sequenced, and four different sequences were confirmed. The four cloned sequences, 100_{—}10.ed, 50_{—}10RS.ed, 500.ed, and 500pRS10_{—}1.ed, are represented in SEQ ID NOs:5, 6, 7, and 8, respectively. The alignment of all four cloned sequences with a portion of the Alu-equivalent consensus sequence (nucleotides 6-84; SEQ ID NO:1) from Haynes et al., is shown in FIG. 1. A new consensus sequence was derived from this alignment (“Majority”), which corresponds to SEQ ID NO:9.

### Example 1.3

### Probe Optimization

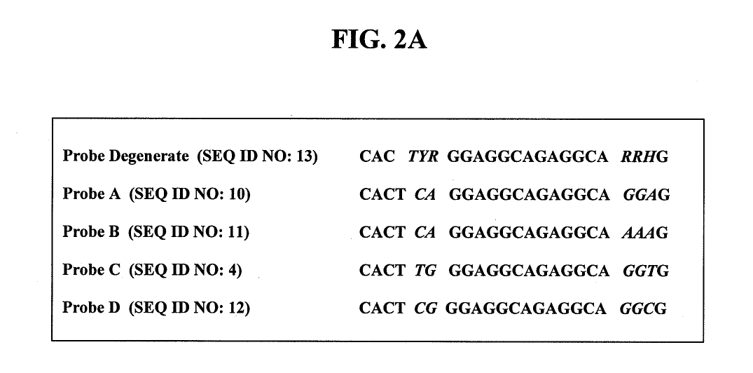

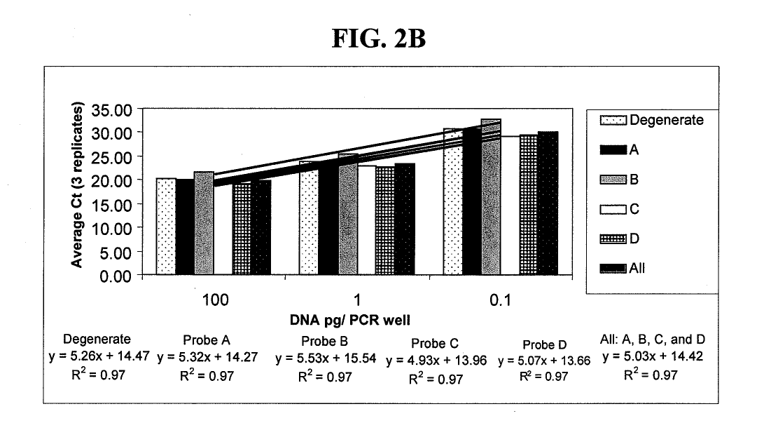

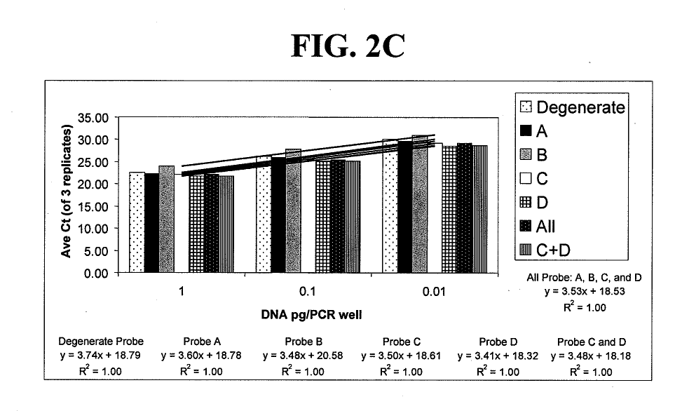

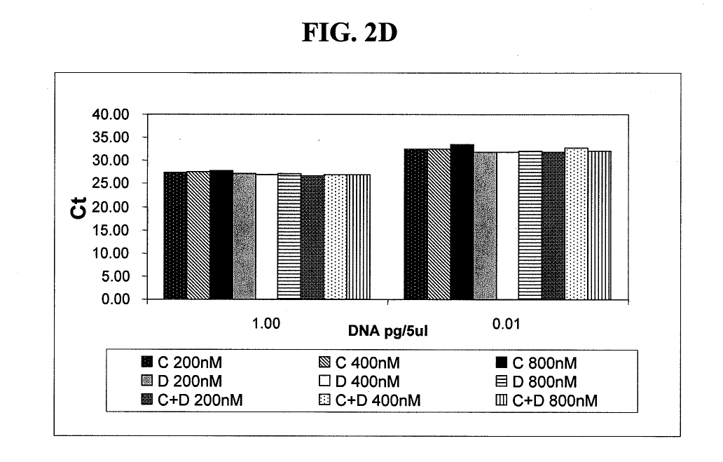

The consensus analysis of Alu-equivalent sequences yielded four nondegenerate probes: Probe A (SEQ ID NO:10), Probe B (SEQ ID NO:11), Probe C (SEQ ID NO:4), and Probe D (SEQ ID NO:12) (FIG. 2A). Based on the sequences of these nondegenerate probes, one degenerate probe was designed, Probe Deg. (SEQ ID NO:13) (FIG. 2A). The probes were designed using the ABI Primer Express Software, and contained a fluorescent reporter dye FAM and a fluorescent quencher, TAMRA.

The aim was to select a probe or a combination of probes that would yield high sensitivity, broad dynamic range, and high signal-to-background ratio. The performance of each of the four nondegenerate probes was compared to each other, to the degenerate probe, as well as to the mixture of 200 nM of each nondegenerate probes. A known concentration of template DNA was extracted from water using the Puregene DNA Purification Kit (Gentra, Minneapolis, Minn.; hereinafter “Gentra”), serially diluted, and subjected to qPCR. The qPCR was run on ABI Prism 7000 TAQMAN® Instrument. As a result of qPCR, a different C_{T} value was obtained for each of the template DNA concentrations (FIG. 2B). Probes were further serially diluted, to determine sensitivity and dynamic range (FIG. 2C). The top performing probes, i.e., the probes with the lowest C_{T} values from FIG. 2C, and the combinations of these probes were titrated at 200 nM, 400 nM, and 800 nM concentrations, and the C_{T} values were subsequently compared in a new qPCR reaction (FIG. 2D). The results indicate that the differences in the various probes were minor and comparable at both the high and the low amounts of the template. Changing probe concentrations did not make significant improvements in C_{T} values and sensitivity. Probe C, however, seemed to have a slightly higher signal-to-background ratio relative to other probes. Probe C at 200 nM was the most consistent across all parameters, and was used in subsequent experimentation.

### Example 1.4

### Optimization of Sample Preparation Conditions

Three extraction methods were tested: Gentra kit, QIAamp DNA Kit (Qiagen, Valencia, Calif.; hereinafter “Qiagen”) and MASTERPURE™ DNA Purification Kit (EPICENTRE®, Madison, Wis.; hereinafter “EpiCentre”).

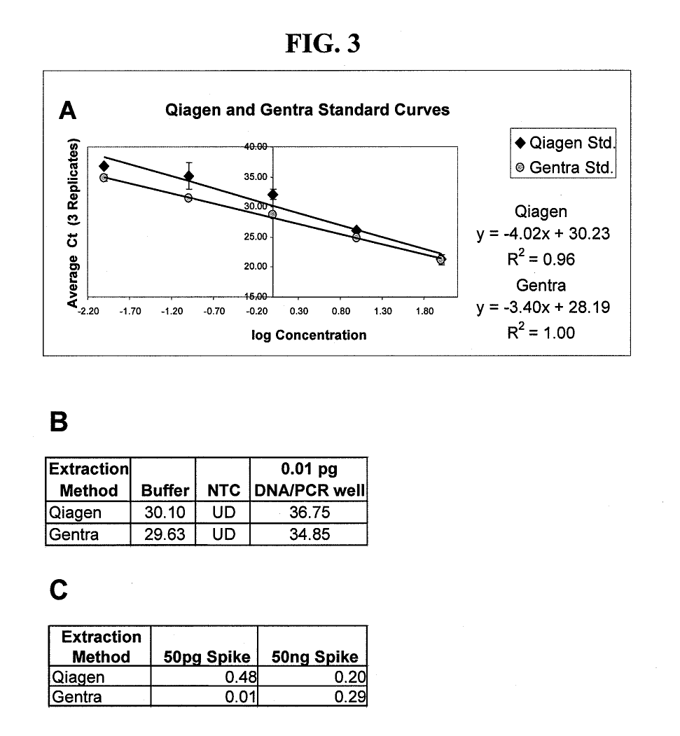

In one experiment, Qiagen versus Gentra extraction methods were compared. The sample was spiked with 50 pg or 50 ng of CHO genomic DNA predigested with Msp I/Kpn I restriction enzymes. Sample and buffer, spiked and unspiked, together with the five-point standard curve, were extracted in parallel using the two kits. The assessment was carried out using purified antibody samples with antibody concentration of 51 mg/mL and buffer (10 mM histidine, 2% sucrose, pH 6.0). The standard curves from both extraction methods are depicted in FIG. 3.

The assay performance was assessed based on spike DNA recovery, % CV and STDEV of PCR replicates, slope and R² of the standard curves, and qualitative assessment of the extraction protocol. Gentra-extracted standard had lower variability between replicates (standard deviation, STDEV), R² closer to 1.00 and slope closer to the optimal slope of −3.26 (FIG. 3A). The sensitivity of the qPCR assay improved when using Gentra rather than Qiagen extraction method. The average of C_{T} values for the Gentra-generated extracted buffer was 29.63. The average of C_{T} values of the Qiagen extracted buffer was 30.10, significantly decreasing the assay's sensitivity (FIG. 3B). The log difference for spike recoveries was also better for samples extracted with Gentra than with Qiagen (FIG. 3C). One major difference in the two methods is that Qiagen uses the spin-column step, whereas Gentra employs isopropyl DNA precipitation with pellet paint coprecipitant. This may account for differences observed in the spike recovery and the standard curve performance. Furthermore, the Qiagen method involved more tube handling, which could increase the risk of contamination.

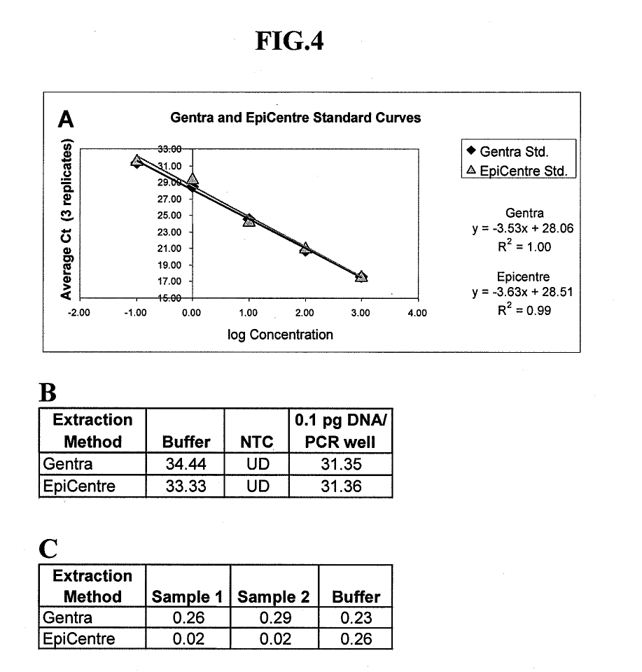

In the second experiment, the Gentra protocol was compared to the EpiCentre protocol (FIG. 4). Samples (both Sample 1 and Sample 2) were spiked with 50 pg of DNA. The EpiCentre outperformed the Gentra protocol. Although the assay's sensitivity was similar for both kits, the log difference of spike recoveries was better for samples extracted with EpiCentre than with Gentra (FIG. 4C). During the DNA precipitation step, DNA pellets generated via the EpiCentre method were tighter and adhered better to the tube after centrifugation than DNA pellets generated via the Gentra method. The differences in Cell Lysis and Protein Precipitation solutions between the two kits could account for differences in pellet formation. Thus, EpiCentre was chosen as the most sensitive, accurate method of DNA preparation.

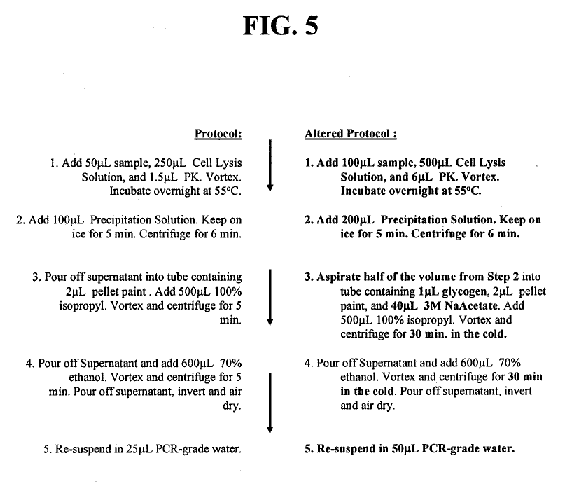

To assure consistency of sample preparation and spike recovery via EpiCentre, the EpiCentre protocol was improved in several steps. In the protein digestion step and the protein precipitation step, the amounts of reagents were doubled. After spinning down the protein pellet, instead of transferring the entire supernatant into a new tube, only half of the total volume (approximately 400 μL) was aspirated to a new tube that contained reagents for DNA precipitation. This had the benefit of establishing more consistent and accurate sample handling (FIG. 5).

The EpiCentre protocol was further optimized to enable it to handle downstream purification process samples or samples in which extraction efficiencies may be affected by high protein concentration and/or low abundance of DNA template. To improve protein digestion, the Proteinase K concentration was doubled to 6 μL/reaction. To facilitate DNA precipitation, glycogen was added and centrifugation during the 100% isopropyl and 70% ethanol wash was performed in the cold at 4° C. All these changes resulted in more consistent extraction results and formation of tighter DNA pellets in various samples and buffer matrices.

### Example 1.5

### Standard Curve Preparation

Two methods for standard curve preparation were compared. In the first method, the each-point-extracted method, all of the dilutions of the standard template were prepared in the respective sample buffer, and then each dilution was extracted individually and each DNA pellet resuspended in PCR-grade water.

In the second method, the single-point-extracted method, only the highest concentration of the standard template was prepared in the sample buffer and subsequently extracted, and then used as a starting concentration for serial dilution in PCR-grade water. Extracting the highest concentration of the standard template (the upper-most-standard curve point) has proven to be a reliable and consistent method for making a five-point standard curve. The single-point extraction method has been tested and has displayed consistent results across different projects and different purification steps.

The differences between the each-point-extracted and the single-point-extracted methods were not significant. Table 1 indicates the slope, the R², and the Y-intercept for the single-point-extracted and the each-point-extracted standard curves for various proteins (e.g., A and B) and protein purification steps (e.g., end product and column 1). The R² values for both methods were within the acceptance criteria of greater or equal to 0.98, the slope for each method was close to the optimal slope of −3.26, and the Y-intercept was comparable across all the experiments. However, the single-point-extracted method is easier to perform and is more reliable and reproducible.

TABLE 1

| | | Slope | | R² | | Y-intercept | |
|---|---|---|---|---|---|---|---|
| | Purification | Single Point | Each Point | Single Point | Each Point | Single Point | Each Point |
| Protein | Step | Extracted | Extracted | Extracted | Extracted | Extracted | Extracted |
| A | End product | −3.38 | −3.03 | 1.00 | 0.98 | 27.72 | 26.95 |
| A | End product | −3.19 | −3.27 | 1.00 | 1.00 | 26.69 | 26.96 |
| A | Column 1 | −3.51 | −3.51 | 0.99 | 1.00 | 28.66 | 28.39 |
| B | End product | −3.38 | −3.48 | 1.00 | 0.99 | 30.70 | 31.38 |

### Example 2

### Optimized Method of Detection and Quantification of Residual Genomic DNA

### Example 2.1

### DNA Preparation with MasterPure DNA Purification Kit

Optimized DNA preparation protocol described in FIG. 5 was used for subsequent experimentation. In the digestion step, 100 μL of sample or buffer was combined with 500 μL of Cell Lysis Solution (EPICENTRE® MasterPure DNA Purification Kit), 100 μg/mL of PCR grade Proteinase K (Roche Diagnostics GmbH, Mannheim, Germany), and 50 ng/mL yeast tRNA (Sigma-Aldrich Co., St. Louis, Mo.) to prevent nonspecific adsorption or loss of DNA. The reactions were incubated overnight at 55° C. in a nonwater incubator. The samples and buffer were spiked with 50 pg of CHO genomic DNA per well, which was previously digested with Msp I and Kpn I restriction enzymes.

After overnight incubation, the tubes were cooled to room temperature. The digested protein was precipitated by adding 200 μL of Protein Precipitation Solution (EPICENTRE® MasterPure DNA Purification Kit), incubated on ice for 5 min and centrifuged for 6 min at 14,000×g, forming a protein pellet on the bottom of the tube. Half (approximately 400 μl) of the supernatant was removed and transferred to a clean tube containing 2 μL pellet paint (Novagen, Darmstadt, Germany), 1 μL of glycogen, and 40 μL 3 M sodium acetate (Novagen). Then 500 μL of isopropyl alcohol was added. The tubes were vortexed and then centrifuged for 30 min at 14,000×g at 4° C. The pellets were washed with 600 μL of 70% ethanol alcohol by inverting the tubes several times and centrifuging for 30 min at 14,000×g in the cold at 4° C. The DNA pellets were air-dried, resuspended in 50 μL of PCR grade water (Roche) and left overnight at 4° C.

### Example 2.2

### qPCR Reaction

The qPCR reaction was set up in a dedicated PCR-preparation-only hood to prevent contamination with template. Each 50 μL reaction consisted of PCR grade water (Roche), 1× Buffer A, 3 mM Mg²⁺, 200 μM dATP, 200 μM dCTP, 200 μM dGTP, 400 μM dUTP, 0.025 U/μL AMPLITAQ® Gold Polymerase, 0.01 U/μL AmpErase UNG, 200 nM probe (FAM-TAMRA), and 900 μM of each forward and reverse primers (SEQ ID NO:2 and SEQ ID NO:3, respectively). The PCR master mix was prepared, and then 45 μL of the master mix was distributed into each well on the 96-well plate. The plate was then moved to the dedicated sample-preparation hood where 5 μL of the template was added to the designated wells. After the plate was sealed and centrifuged briefly, it was run on TAQMAN® ABI Prism 7000 instrument (BioReliance, Rockville, Md.) using the standard thermocycle protocol, which consisted of: (1) an initial step (UNG treatment) at a temperature of 50° C. for 2 minutes, (2) an UNG deactivation step and polymerase activation step at 95° C. for 10 minutes, and (3) 45 cycles of denaturing at a temperature of 95° C. for 15 seconds and annealing/extension at a temperature of 60° C. for 1 minute.

### Example 2.3

### Detection of Residual Genomic DNA Contamination in the Optimized Protocol

Several experiments were run according to the optimized assay protocol. Table 2 indicates the slope, R², and the average (n=3) C_{T} values for the assay LOD (defined as the average signal of the extracted buffer) and the LOQ (defined as the average signal of the last point on the standard curve) for various proteins (e.g., A and B) and protein purification steps (e.g., end product, load, column 1, column 2, and column 3).

This optimized assay for testing residual CHO DNA in a protein sample has been proven to work across different purification steps and projects. Based on the slope and R² of the standard curve, limit of detection (LOD) and sensitivity (Limit of Quantitation, LOQ), the developed qPCR protocol performed consistently and in a robust manner. The results indicated that the slope of the standard curve consistently came close to the optimal slope of −3.26 with a narrow range of −3.25 to −3.58, and the R² consistently met the acceptance criteria of greater or equal to 0.98. The LOQ (at 50 pg/mL) criterion requires that the average C_{T} of the last point on the standard curve (LOQ) must be less than the C_{T} of the extracted buffer. All of the attempted experiments passed the criterion set forth for LOQ.

TABLE 2

| | | | | LOD | LOQ 50 pg/mL |
|---|---|---|---|---|---|
| Protein | Step | Slope | R² | Buffer CT | Sensitivity CT |
| A | Column 1 | −3.52 | 0.99 | 34.45 | 30.80 |
| A | Column 2 | −3.51 | 1.00 | 35.88 | 32.67 |
| A | Column 3 | −3.45 | 1.00 | 32.06 | 30.37 |
| B | End product | −3.54 | 0.99 | 34.01 | 32.17 |
| A | End product | −3.48 | 0.99 | 43.23 | 31.29 |
| A | Load | −3.53 | 0.99 | 37.75 | 31.40 |

What is claimed is:

1. A method of detecting contaminant genomic DNA in a sample comprising:
(a) purifying genomic DNA from the sample;
(b) adding a pair of oligonucleotide primers that are complementary to repetitive sequences of the genomic DNA;
(c) amplifying the repetitive sequences with the pair of oligonucleotide primers using a real time PCR amplification method; and
(d) detecting the presence of amplified repetitive sequences,
wherein detection of the amplified repetitive sequences indicates the presence of the contaminant genomic DNA in the sample.

2. The method of claim 1, wherein the real time PCR amplification method is a quantitative real time PCR amplification method.

3. The method of claim 2, wherein the quantitative real time PCR amplification method is TAQMAN® Probe technology.

4. A method of detecting contaminant genomic DNA in a sample comprising:
(a) purifying genomic DNA from the sample;
(b) adding both a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on an opposite end;
(c) amplifying the repetitive sequences using a nucleic acid polymerase having 5′ to 3′ exonuclease activity; and
(d) measuring the change in fluorescence of the sample during amplification,
wherein the change in fluorescence indicates detection of amplified repetitive sequences and correlates with the presence of the contaminant genomic DNA in the sample.

5. The method of claim 4, wherein the step of purifying genomic DNA from the sample comprises:
(a) digesting protein and RNA in the sample; and
(b) extracting total DNA from a sample by precipitation.

6. The method of claim 5, wherein the step of purifying genomic DNA from the sample comprises using MASTERPURE™ DNA Purification Kit.

7. The method of claim 4, wherein the pair of oligonucleotide primers comprises the nucleic acid sequences of SEQ ID NO:2 and SEQ ID NO:3.

8. The method of claim 4, wherein the oligonucleotide probe comprises nucleic acid sequences selected from the group consisting of SEQ ID NO:4, SEQ ID NO:10, SEQ ID NO:11, SEQ ID NO:12, and SEQ ID NO:13.

9. The method of claim 8, wherein the oligonucleotide probe comprises nucleic acid sequence of SEQ ID NO:4.

10. The method of claim 4, wherein the fluorescent reporter is FAM and the quencher dye is TAMRA.

11. The method of claim 4, wherein the repetitive sequences are Alu sequences or Alu-equivalent sequences.

12. The method of claim 4, wherein the pair of oligonucleotide primers and the oligonucleotide probe are designed based on a consensus of several Alu sequences or Alu-equivalent sequences of an organism.

13. The method of claim 4, wherein the nucleic acid polymerase is Taq polymerase.

14. The method of claim 4, wherein the step of amplifying further comprises a step of monitoring for sample recovery and assay performance, wherein the step of monitoring comprises adding a known genomic DNA spike to the sample.

15. The method of claim 14, wherein the known genomic DNA spike has a genomic DNA concentration of 10 ng/mL.

16. The method of claim 4, wherein the genomic DNA is CHO cell genomic DNA.

17. The method of claim 4, wherein the sample comprises a purified protein.

18. The method of claim 17, wherein the sample is a pharmaceutical composition, and wherein the purified protein is a therapeutic protein.

19. A method of quantifying contaminant genomic DNA in a first sample comprising:
(a) purifying genomic DNA from the first sample;
(b) adding to the first sample a pair of oligonucleotide primers that are complementary to repetitive sequences of the genomic DNA;
(c) adding to a second sample, comprising a known amount of genomic DNA, a pair of oligonucleotide primers that are complementary to repetitive sequences of the genomic DNA;
(d) amplifying repetitive DNA sequences in the first and second samples using a real time PCR amplification method; and
(e) determining from the amplified repetitive DNA sequences of the second sample the amount of the contaminant genomic DNA in the first sample.

20. The method of claim 19, wherein the real time PCR amplification method is a quantitative real time PCR amplification method.

21. The method of claim 20, wherein the quantitative real time PCR amplification method is TAQMAN® Probe technology.

22. A method of quantifying contaminant genomic DNA in a first sample comprising:
(a) purifying genomic DNA from the first sample;
(b) adding to the first sample a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on an opposite end;
(c) adding to a second sample, comprising a known amount of genomic DNA, a pair of oligonucleotide primers that are complementary to repetitive sequences of the genomic DNA and an oligonucleotide probe capable of hybridizing to the repetitive sequences 3′ relative to one of the pair of oligonucleotide primers, said probe containing a fluorescent reporter on one end and a quencher dye on an opposite end;
(d) amplifying repetitive DNA sequences in the first and second samples using a nucleic acid polymerase having 5′ to 3′ exonuclease activity;
(e) measuring the change in fluorescence of the first and second samples during amplification;
(f) comparing the change in fluorescence of the first and second samples; and
(g) determining from the comparison of fluorescence of the first and second samples the amount of contaminant genomic DNA in the first sample.

22. The method of claim 21, wherein the step of purifying genomic DNA from the first sample comprises:
(a) digesting protein and RNA in the first sample; and
(b) extracting total DNA from the first sample by precipitation.

23. The method of claim 22, wherein the step of purifying genomic DNA from the first sample comprises using MASTERPURE™ DNA Purification Kit.

24. The method of claim 21, wherein the pair of oligonucleotide primers comprises nucleic acid sequences of SEQ ID NO:2 and SEQ ID NO:3.

25. The method of claim 21, wherein the oligonucleotide probe comprises nucleic acid sequences selected from the group consisting of SEQ ID NO:4, SEQ ID NO:10, SEQ ID NO:11, SEQ ID NO:12, and SEQ ID NO:13.

26. The method of claim 25, wherein the oligonucleotide probe comprises nucleic acid sequence of SEQ ID NO:4.

27. The method of claim 21, wherein the fluorescent reporter is FAM and the quencher dye is TAMRA.

28. The method of claim 21, wherein the repetitive sequences are Alu sequences or Alu-equivalent sequences.

29. The method of claim 21, wherein the pair of oligonucleotide primers and the oligonucleotide probe are designed based on the consensus of several Alu sequences or Alu-equivalent sequences of an organism.

30. The method of claim 21, wherein the nucleic acid polymerase is Taq polymerase.

31. The method of claim 21, wherein the known amount of genomic DNA in the second sample is predigested with Msp I or Kpn I restriction enzymes.

32. The method of claim 21, wherein the genomic DNA is a CHO cell genomic DNA.

33. The method of claim 21, wherein the first sample comprises a purified protein.

34. The method of claim 33, wherein the first sample is a pharmaceutical composition, and wherein the purified protein is a therapeutic protein.

35. The method of claim 34, wherein the amount of contaminant genomic DNA in the pharmaceutical composition is less than 10 nanograms per dose.


SEQUENCE LISTING (13 sequences)

SEQ ID NO: 1 - DNA, 79 bp, artificial
  Consensus of several CHO-K1 cell Alu-equivalent sequences
cattggtggcacacacctttagtcccagcactcaggaggcagaggcaggaggatcacttgagttcaagagccagcctgg

SEQ ID NO: 2 - DNA, 22 bp, artificial
  Forward qPCR primer
ttggtggcacacacctttaatc

SEQ ID NO: 3 - DNA, 23 bp, artificial
  Reverse qPCR primer
gctggctcttgaactcacagaga

SEQ ID NO: 4 - DNA, 23 bp, artificial
  qPCR probe
cacttgggaggcagaggcaggtg

SEQ ID NO: 5 - DNA, 78 bp, Cricetulus griseus
cattggtggcacacacctttaatcccagcacttgggaggcagaggcaggtggatctgtgagttcaagagccagcctgg

SEQ ID NO: 6 - DNA, 79 bp, Cricetulus griseus
cagagatgcacctgcctcttcttctgagtgctgggtttaaaggtgtgtgccaccaatgccagattgtcggctgctctag

SEQ ID NO: 7 - DNA, 80 bp, Cricetulus griseus
cattggtggcacacacctttaatcccagcactcaggaggcagaggcaaaaggatctctgtgagttcaagagccagcctgg

SEQ ID NO: 8 - DNA, 80 bp, Cricetulus griseus
gattggtggcacacacctttaatcccagcactcgggaggcagaggcaggcgaatctctgtgagttcaagagccagcctgg

SEQ ID NO: 9 - DNA, 80 bp, artificial
  Consensus of several CHO-K1 cell Alu-equivalent sequences
cattggtggcacacacctttaatcccagcactcgggaggcagaggcaggtggatctctgtgagttcaagagccagcctgg

SEQ ID NO: 10 - DNA, 23 bp, artificial
  qPCR probe
cactcaggaggcagaggcaggag

SEQ ID NO: 11 - DNA, 23 bp, artificial
  qPCR probe
cactcaggaggcagaggcaaaag

SEQ ID NO: 12 - DNA, 23 bp, artificial
  qPCR probe
cactcgggaggcagaggcaggcg

SEQ ID NO: 13 - DNA, 23 bp, artificial
  qPCR probe
cactyrggaggcagaggcarrhg